(19) United States
(12) Patent Application Publication
(10) Pub. No.: US 2007/0191777 A1
(43) Pub. Date: Aug. 16, 2007

(54) Enclosed Needle Device with Fluid Path Access

(75) Inventor: Alan D. King, Burlington, CT (US)

Correspondence Address: WOOD, HERRON & EVANS, LLP, 2700 CAREW TOWER, 441 VINE STREET, CINCINNATI, OH, 45202, US

(73) Assignee: MEDEX, INC., Carlsbad, CA (US)

(21) Appl. No.: 11/276,154

(22) Filed: Feb. 16, 2006

(51) Int. Cl. A61M 5/178

(52) U.S. Cl. 604/164.08; 128/919

(57) ABSTRACT

An enclosed needle catheter insertion device (10) includes an elongated needle guard housing (20) and an elongated needle support housing (22). The support housing (22) is received for movement in guard housing (20) to pull needle (24) in from catheter (12) to be enclosed by guard housing (20). Support housing (22) is of sufficient length that a proximal end (42) thereof is accessible at or beyond a proximal end (28) of guard housing (20) even with the needle (24) extended out of catheter (12) for use, so as to allow for access to the fluid path (44) of support housing (22) to needle lumen (49).

### FIELD OF THE INVENTION

The present invention relates to needle insertion devices, and more particularly, to an enclosed needle catheter insertion device.

### DESCRIPTION OF PRIOR ART

Over-the-needle catheters are well known in the art. In such devices, a cannula needle projects through a catheter tube with its sharp tip projecting out of the end of the tube. The sharp tip of the needle is used to pierce the skin and the blood vessel so as to carry the end of the catheter into the vessel. Once in place, the needle is withdrawn, leaving the catheter in place for administration or withdrawal of fluids, such as by connection with the now-exposed catheter hub.

In order to reduce the risks of accidental needle sticks after the needle has been removed from the catheter, various proposals have been made to shield the needle tip. One class of devices intended to shield the needle tip includes a needle guard housing into which the needle is received as it is pulled out from the catheter. The guard housing may include as part of its distal end a nose similar to a male slip luer that is adapted to be removably held to the catheter hub. The guard housing is of sufficient length that it essentially encloses the entire length of the needle therein, thus shielding the needle tip. In some cases, the sharp tip is fully inside the guard housing, while in others, the sharp tip may be inside the nose to thus maintain alignment of the needle. In either setting, however, the needle is considered to be enclosed and the tip shielded. To that end, the needle is supported by a needle support hub or housing within the guard housing and which is movable relative to the guard housing from a first position at which the distal end of the support housing is positioned toward the distal end of the guard housing with the needle extending out of the guard housing (and through and out of the catheter when the guard housing is held to the catheter hub), to a second position with the distal end of the support housing positioned away from the distal end of the guard housing so as to withdraw the needle to be enclosed by the guard housing.

The support housing might be spring biased to automatically move the needle into the second position when a latch is activated as shown, for example, in U.S. Pat. No. 4,747,831 and in the commercially available AutoGuard shielded IV catheter from Becton Dickinson and Company. Or the support housing may be manually moved to the second position such as by manipulation of walls or wings of or attached to the support housing. In the manual type of device, a projection and slot detent mechanism on the housings cooperate to retain the housings with the needle in the second position enclosed in the guard housing thus shielding the needle tip. An example of such a manual device is the highly successful PROTECTIV Safety I.V. Catheter marketed by Medex, Inc., the assignee hereof. After moving into the second, shielded position of the needle, the guard housing may be removed from the catheter hub and discarded with the needle shielded therein, leaving the catheter hub accessible as necessary.

### SUMMARY OF THE INVENTION

While devices that use a housing to shield the needle tip by enclosing the needle have been well-accepted and are in widespread use, further improvements are desired. By way of example, in many clinical settings, it is desirable to access the fluid path to the lumen of the needle. Such access is desired for flushing, prior to or during use, or aspiration with a syringe, or for allowing the use of a guidewire during insertion, for example. In commercially available enclosed needle devices such as the AutoGuard or the PROTECTIV devices, however, the needle support housing is quite short in length, sufficient only to support the needle and/or provide a flashback chamber, but otherwise generally confined within the needle guard housing. As a consequence, in the first position, the support housing is recessed well within the guard housing and generally not accessible for attachment of a syringe to flush or aspirate through the needle lumen, or for insertion of a guidewire therein, for example. Further, in the second position, the support housing may still be within the confines of the guard housing and thus still not easily accessible for such purposes.

The present invention provides an enclosed needle catheter insertion device which facilitates fluid or guidewire access to the fluid path to the needle lumen such as to allow for flushing, aspiration, guidewire access, or the like. To that end, and in accordance with the principles of the present invention, the support housing is provided as an elongated housing such that the proximal portion thereof extends out of the guard housing in at least the first position of the needle thereby rendering the proximal portion of the support housing accessible to the medical practitioner. The proximal portion may include or define an access port for use by the medical practitioner to access the fluid path.

Advantageously, the guard and support housings telescope along a common axis, such that the proximal portion of the support housing is accessible at a proximal portion of the guard housing. In that case, the support housing is at least about as long as the guard housing such that the proximal portion will be accessible to the medical practitioner at all times, irrespective of the position of the housing (i.e., whether in the first or second needle positions, or even therebetween).

Further advantageously, a flash plug is associated with the proximal portion of the support housing, and may be removably fitted into the flushing port thereof.

By virtue of the foregoing, there is thus provided an enclosed needle catheter insertion device which facilitates access to the fluid path to the needle lumen. These and other objects and advantages of the present invention shall be made apparent from the accompanying drawings and the description thereof.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is a perspective view of an enclosed needle catheter insertion device which facilitates fluid path access in accordance with the principles of the present invention;

FIGS. 2A and 2B are cross-section views of the enclosed needle catheter insertion device of FIG. 1 in respective first and second positions for purposes of explaining the principles of the present invention;

FIGS. 3A and 3B are bottom views, the latter partially cut-away, of the enclosed needle catheter insertion device of FIG. 1 in respective first and second positions for purposes of explaining the principles of the present invention; and

FIGS. 4 and 5 are cross-section views of alternative nose and catheter hub arrangements which may be used with the present invention.

### DETAILED DESCRIPTION OF THE DRAWINGS

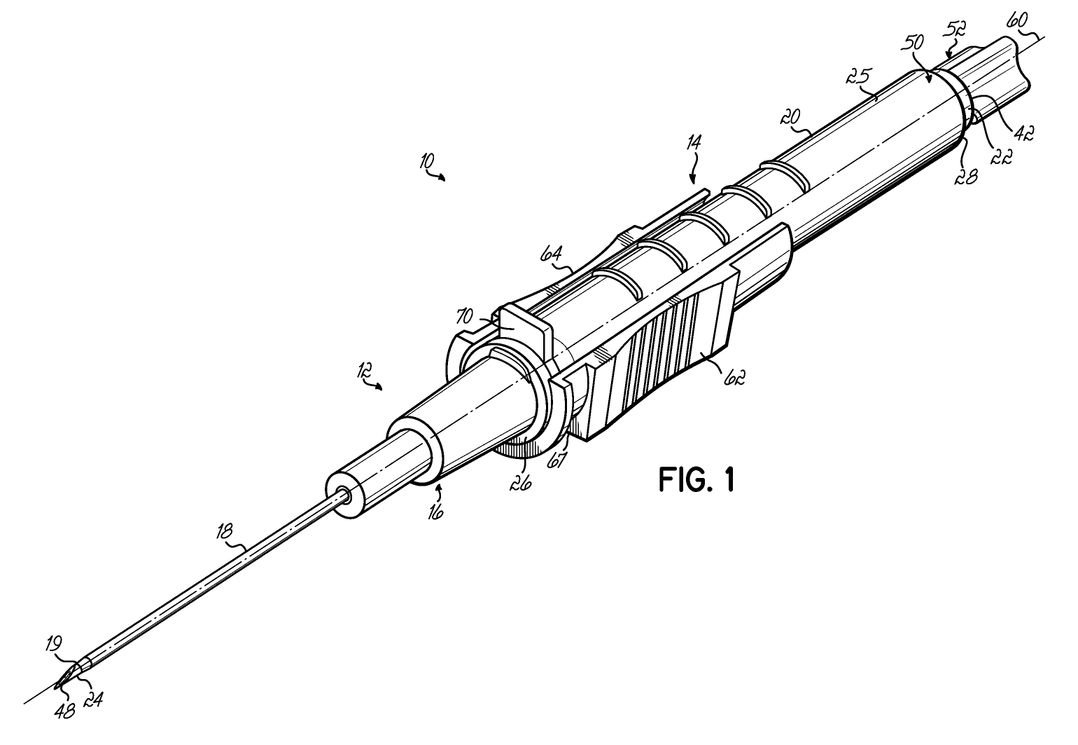

One embodiment 10 of an enclosed needle catheter insertion device in accordance with the principles of the present invention is shown in accompanying FIGS. 1 through 3B (which are not necessarily to scale in order to show the various components more readily). Catheter insertion device 10 includes a catheter 12, and a needle insertion assembly 14 including features of the present invention. Catheter 12 includes a catheter hub 16 having an inner wall 17 corresponding to a female luer taper, and a catheter tube 18 held to the catheter hub 16 by any available means (such as an eyelet 15) and extending distally therefrom to its distal, advantageously beveled, end 19. Needle insertion assembly 14 includes an elongated, advantageously cylindrical, outer needle guard housing 20; an elongated, advantageously cylindrical inner needle support chamber or housing 22 telescopingly received in guard housing 20; and a needle 24.

Guard housing 20 has a sidewall 25 extending between distal end or cap 26 and proximal end 28 and defining therewithin a space 30 through which support housing 22 moves and into which needle 24 is ultimately received to be enclosed as will be described below. Distal end 26 includes a nose 32, which may be a male luer taper sized to be removably fitted within the catheter hub 16 and to mate to the inner, female luer taper wall 17 thereof. Housing 20 includes a longitudinal notch 34 running along a portion of its length between ends 26 and 28 for purposes to be described hereinafter.

Support housing 22 has a sidewall 38 extending between distal end 40 and proximal end 42 and defining therebetween a fluid path or lumen 44. Needle 24 is supported by, and may advantageously be affixed to, support housing 22 such that the shaft 46 of needle 24 extends distally from distal end 40 of support housing 22 to a sharp tip 48. The fluid path 44 of support housing 22 is in fluid communication with the lumen 49 of needle 24 such that blood (not shown) may flash back through needle 22 into the fluid path 44 whereby housing 22 also serves as a flash chamber. In that respect, the elongated nature of support housing 22 provides improved flashback visual confirmation. In the embodiment 10 shown herein, housings 20 and 22 are advantageously of about equal length such that the proximal end 42 of support housing 22 is accessible at or beyond the proximal end 28 of guard housing at all times. Support housing 22 could be a bit shorter but still accessible at or beyond proximal end 28 of guard housing 20 due to a cutout (not shown) in guard housing 20 to move proximal end 28 inwardly with a proximal, partial extension (not shown) of guard housing 20 defining an awning or cover (also not shown). A fluid path access port 50 is defined at the proximal end 42 of support housing 22 through which a medical practitioner (not shown) may access fluid path 44 to introduce or withdraw (aspirate) fluids (not shown) through needle lumen 49, such as with a syringe (not shown), or to introduce a guidewire (not shown) therethrough.

To prevent blood (not shown) from exiting support housing 22 during flashback, a flash plug 52 including material 54 adapted to pass air but not blood or other fluids is advantageously associated with support housing 22 near the proximal end 42 thereof. In the embodiment 10 shown herein, flash plug 52 includes a plug housing 56 to hold the material 54 and to define an insert end 58 of male luer taper shape adapted to be frictionally fitted into access port 50 which may advantageously have a mating female luer taper as at 59. Due to the frictional fit therebetween, flash plug 52 normally stays in place closing up flush port 50 against passage of fluids therethrough, but plug 52 can be pulled out giving the medical practitioner access to port 50 for purposes of access to fluid path 44 to needle lumen 49.

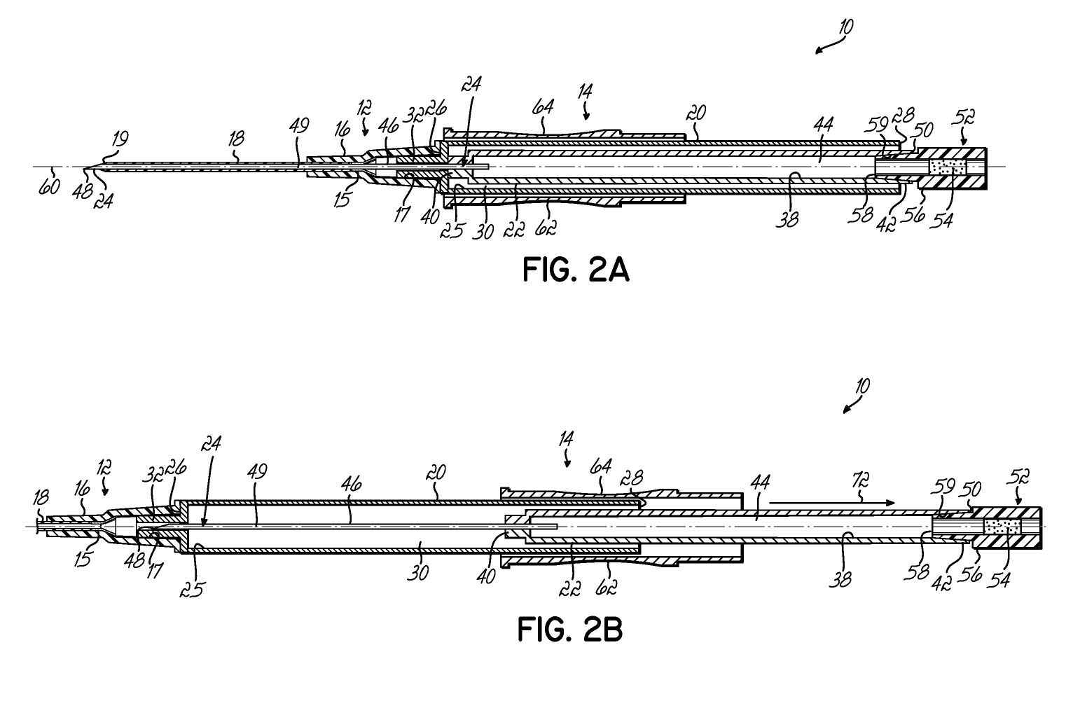

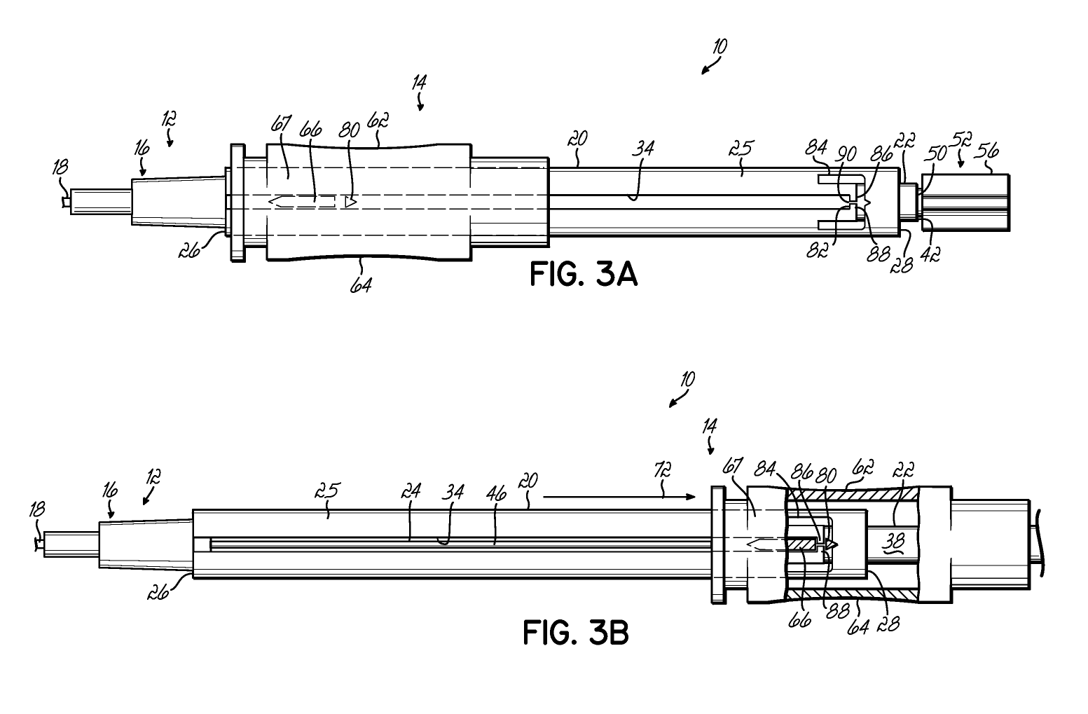

Housings 20 and 22 are telescopingly received such that one may move relative to the other along a common axis 60. To that end, a pair of gripping wings 62, 64 are positioned adjacent but outside of guard housing sidewall 25. A rib 66 extends through lateral notch 34 and joins plate 67 supporting gripping wings 62, 64 to sidewall 38 of support housing 22. A medical practitioner may pull on gripping wings 62, 64 to cause relative motion between housings 20 and 22 from a first position of needle 24 that will be described in connection with FIGS. 2A and 3A, to a second position of needle 24 that will be described with reference to FIGS. 2B and 3B. The wings 62, 64 may also be joined across the top to, in effect, create a tubular member (not shown) about guard housing 20. The tubular member may be elongated (either as one cylinder or by addition of a cap portion, for example) to match the length of guard housing 20.

In the first position of needle 24, shown in FIGS. 2A and 3A, with the distal ends 26 and 40 of housings 20 and 22 positioned towards, and possibly in contact with, each other, needle 24 extends out of the distal end 26 of guard housing 20 and through catheter tube 18, to position sharp needle tip 48 beyond distal end 19 of catheter tube 18. In the first position of needle 24, it will be appreciated that insertion of catheter tube 18 into a patient's blood vessel (not shown) is accomplished by grasping wings 62 and 64 between the thumb and second finger, for example, with the sharp needle tip 48 angled against the patient's skin (not shown) and pushing the entire device distally (to the left in FIG. 2) so as to drive the needle 24 and catheter tube 18 into the patient (not shown). Flashback of blood (not shown) may appear in the flashback chamber defined by fluid path 44 of support housing 22.

Due to the elongated nature of support housing 22 in accordance with the principles of the present invention, proximal end 42 of housing 22 is accessible to the medical practitioner at or beyond proximal end 28 of guard housing 20 in at least the first position shown in FIGS. 2A and 3A. Thus, if desired, the medical practitioner (not shown) may, after removal of flash plug 52 (or before it is attached), preflush the device, attach a syringe or load a guidewire (both not shown) prior to insertion. Also, if desired or necessary, during the process of insertion of device 10 and/or after catheter tube distal end 19 is positioned in the vessel (not shown), the medical practitioner (not shown) may remove flash plug 52 and flush or aspirate device 10 through access port 50. Flash plug 52 may then be put back in place when access is no longer required.

After catheter distal end 19 is positioned as desired, needle 24 is to be withdrawn from catheter 12 by causing support housing 22 to move to a second needle position in which the needle shaft 46 and sharp tip end 48 are enclosed by guard housing 20. To this end, the medical practitioner, using one or two hands as desired, pulls on wings 62, 64, possibly by leveraging against push-off tab 70 formed at the distal end 26 of guard housing 20, so as to cause the distal ends 26 and 40 of housings 20 and 22 to move from the position where they are towards each other as seen in FIGS. 2A and 3A to a position where they are away from each other (represented by movement of support housing 22 to the right along arrow 72 in FIGS. 2B and 3B) as seen in FIGS. 2B and 3B. At any time during the traverse from the first to the second position, the medical practitioner (not shown) may access fluid path 44 through access port 50 if desired or necessary. In the second position, needle shaft 46 is within space 30 of guard housing 20 and sharp needle tip 48 is within nose 32, if not all the way into space 30, such that needle 24 is enclosed by guard housing 20.

As the needle 24 moves into the second position, housings 20 and 22 lock into the second needle position by cooperation of lock structure on each of the housings. To that end, a projection 80 coupled to support housing 22 such as on the rib 66 thereof, rides through longitudinal notch 34 of housing 20 until it reaches the proximal end 82 of notch 34. At the proximal end 82 of notch 34, an outer, U-shaped notch 84 is provided to define fingers 86, 88 thereat. Projection 80 is shaped to cam apart the fingers 86, 88 through a slot 90 therebetween, so that projection 80 may pass into U-shaped notch 84, whereafter, fingers 86, 88 snap back together locking projection 80 in place and holding the housings 20 and 22 in the second needle position with needle 24 enclosed within guard housing 20. Guard housing 20 may be removed from catheter 12 by a pulling or twisting motion, leaving hub 16 exposed for use by the medical practitioner. The needle insertion assembly 14 remains in the second needle position and may be discarded.

Although not shown herein, in another embodiment, a spring may be included within guard housing 20 which is biased to urged support housing distal end 40 away from guard housing distal end 26. With that embodiment, after placement of catheter tip 19, the medical practitioner may activate a release mechanism which will activate the spring and cause the needle to be moved automatically to the second position. In that embodiment, wings 62 and 64 and related structure may be dispensed with. But the support housing 22 is elongated to be accessible at its proximal end 42 even in this embodiment in accordance with the principles of the present invention.

By virtue of the foregoing, there is thus provided an enclosed needle catheter insertion device which facilitates access to the fluid path to the needle lumen.

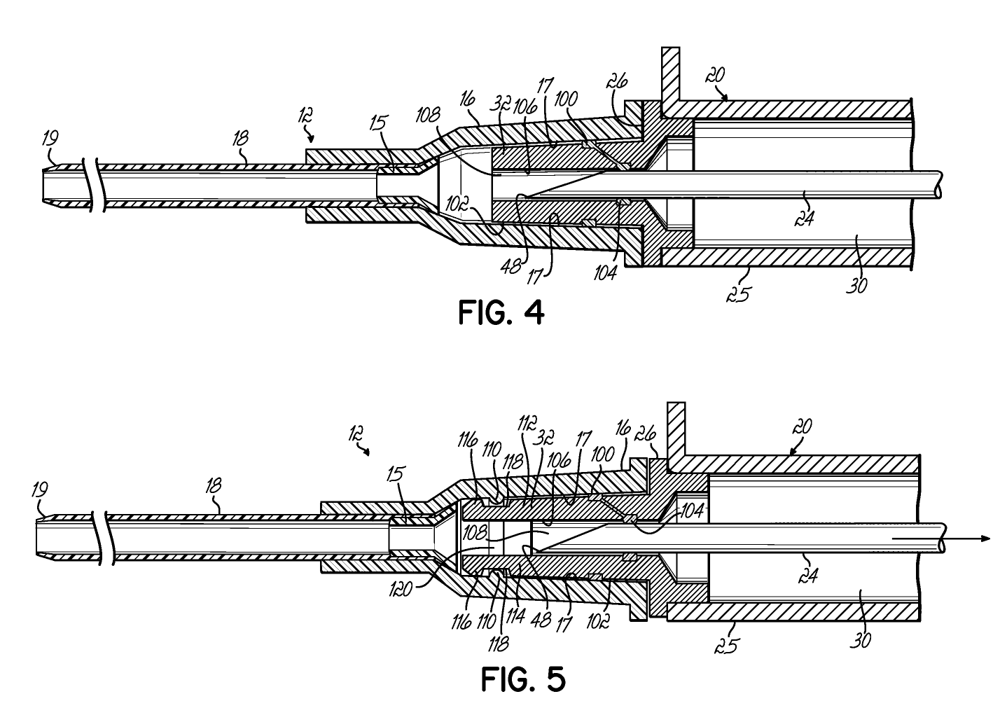

While the present invention has been illustrated by the description of embodiments thereof, and while the embodiments have been described in considerable detail, it is not intended to restrict or in any way limit the scope of the appended claims to such detail. Additional advantages and modifications will readily appear to those skilled in the art. For example, housing 20 and or 22 could be shaped other than cylindrical, including rectangular. Further, the housings 20 and 22 may be held in or moved into the second position of the needle 24 in other ways than as described herein. Nose 32 could include one or more internal or external seals such as co-molded elastomeric gasket 100 integrally associated with exterior wall 102 of nose 32 to form a seal with the inner wall 17 of catheter hub 16 and/or co-molded elastomeric gasket 104 integrally associated with inner wall 106 defining passageway 108 through which needle 24 is received to form a seal therewith as shown in FIG. 4 and as more particularly shown and described in the commonly assigned and concurrently filed U.S. patent application entitled “Sealing Catheter Hub Attachment”, Attorney Docket No. MDXVA-104US. Further or alternatively, nose 32 and catheter hub 16 could be modified to provide a duckbill release mechanism as shown in the commonly assigned and concurrently filed U.S. patent application entitled “Enclosed Needle Device with Duckbill Release Mechanism”, Attorney Docket No. MDXVA-94US. To that end, an annular rib 110 (or multiple segments thereof) project from inner wall 17 of hub 16 and a pair of arms 112, 114 extend distally from nose 32 and are adapted to flex. One or both of arms 112, 114 may include a detent 116 defining a recess 118 therebehind with detent 116 normally extending to an outer diametrical distance larger than the inner diameter defined at rib 110 so as to form a releasable hub attachment with rib 110 in recess 118 when needle 24 is not in the space 120 between arms 112 and 114. Nose 32 may also include the integral co-molded elastomeric seals 100 and 104 as described in connection with FIG. 4. Alternatively, although not as desirable, rib 110 could be a recess into wall 17, with detent(s) 116 having a projecting rib on the end of arm 112 and/or 114, and without the recess 118. The disclosures of both of the aforementioned commonly assigned and concurrently filed U.S. patent applications (Attorney Docket Nos. MDXVA-104US and MDXVA-94US) are incorporated herein by reference as if fully set out herein. The invention in its broader aspects is, therefore, not limited to the specific details, representative apparatus and method, and illustrative examples shown and described. Accordingly, departures may be made from such details without departing from the spirit or scope of the general inventive concept.

Having described the invention, what is claimed is:

1. An enclosed needle catheter insertion device comprising:
a catheter having a catheter hub and a catheter tube extending distally from the catheter hub;
an elongated needle guard housing extending between a distal end removably coupled to the catheter hub and a proximal end defining a space therebetween;
an elongated needle support housing telescopingly received within the space of the needle guard housing, the needle support housing having a distal end and a proximal end defining a fluid path therebetween;
a needle extending distally from the support housing distal end and extending through the catheter tube such that a sharp tip of the needle is exposed in a first position of the needle with the distal end of the support housing positioned toward the distal end of the guard housing, the needle being withdrawn from the catheter and enclosed by the guard housing in a second position of the needle with the distal end of the support housing positioned away from the distal end of the guard housing; and
the support housing being elongated sufficiently that a proximal portion thereof is accessible at or beyond the proximal end of the guard housing in at least the first position of the needle.

2. The catheter insertion device of claim 1 further comprising a fluid path access port in the proximal portion of the support housing.

3. The catheter insertion device of claim 2 further comprising a flash plug removably fitted in the access port.

4. The catheter insertion device of claim 2, the access port having a female luer taper.

5. The catheter insertion device of claim 4 further comprising a flash plug having a male luer taper removably fitted in the female luer taper of the access port.

6. The catheter insertion device of claim 1 further comprising a fluid path access port associated with the proximal portion of the support housing.

7. The catheter insertion device of claim 1, the proximal end of the support housing being accessible.

8. The catheter insertion device of claim 7 further comprising a fluid path access port at the proximal end of the support housing.

9. The catheter insertion device of claim 8 further comprising a flash plug removably fitted in the fluid path access port.

10. The catheter insertion device of claim 7, the access port having a female luer taper.

11. The catheter insertion device of claim 10 further comprising a flash plug having a male luer taper removably fitted in the female luer taper of the access port.

12. The catheter insertion device of claim 1 further comprising cooperating lock structure associated with the guard and support housings.

13. The catheter insertion device of claim 12 wherein the cooperating lock structure includes a projection and a slot associated with respective ones of the needle guard housing and the needle support housing.

14. The catheter insertion device of claim 1, the support housing being elongated sufficiently that the proximal portion thereof extends out of the guard housing in at least the first position of the needle.

15. The catheter insertion device of claim 1, the guard housing and the support housing being telescoped along a common axis.

16. The catheter insertion device of claim 1, the distal end of the guard housing including a nose, the sharp tip of the needle extending beyond the nose in the first position of the needle and being within the nose in the second position of the needle.

17. The catheter insertion device of claim 1, the distal end of the guard housing including a nose adapted to fit into the catheter hub and through which the needle is received, the nose including an elastomeric gasket integrally associated therewith.

18. The catheter insertion device of claim 1 further comprising a duckbill release mechanism.

19. A needle guard for a catheter insertion device comprising:
an elongated needle guard housing extending between a distal end and a proximal end and defining a space therebetween;
an elongated needle support housing telescopingly received within the space of the needle guard housing, the needle support housing having a distal end and a proximal end defining a fluid path therebetween;
a needle extending distally from the support housing distal end and having a sharp tip extending out beyond the distal end of the guard housing in a first position of the needle with the distal end of the support housing positioned toward the distal end of the guard housing, the needle being enclosed by the guard housing in a second position of the needle with the distal end of the support housing positioned away from the distal end of the guard housing; and
the support housing being elongated sufficiently that a proximal portion thereof is accessible at or beyond the proximal end of the guard housing in at least the first position of the needle.

20. The needle guard of claim 19 further comprising a fluid path access port in the proximal portion of the support housing.

21. The needle guard of claim 20 further comprising a flash plug removably fitted in the access port.

22. The needle guard of claim 19, the access port having a female luer taper.

23. The needle guard of claim 22 further comprising a flash plug having a male luer taper removably fitted in the female luer taper of the access port.

24. The needle guard of claim 19 further comprising a fluid path access port associated with the proximal portion of the support housing.

25. The needle guard of claim 19, the proximal end of the support housing being accessible.

26. The needle guard of claim 25 further comprising a fluid path access port at the proximal end of the support housing.

27. The needle guard of claim 26 comprising a flash plug removably fitted in the access port.

28. The needle guard of claim 19 further comprising cooperating lock structure associated with the guard and support housings.

29. The needle guard of claim 28 wherein the cooperating lock structure includes a projection and a slot associated with respective ones of the needle guard housing and the needle support housing.

30. The needle guard of claim 19, the support housing being elongated sufficiently that the proximal portion thereof extends out of the guard housing in at least the first position of the needle.

31. The needle guard of claim 19, the guard housing and the support housing being telescoped along a common axis.

32. The needle guard of claim 19, the distal end of the guard housing including a nose, the sharp tip of the needle extending beyond the nose in the first position of the nose and being within the nose in the second position of the needle.

33. The needle guard of claim 19, the distal end of the guard housing including a nose adapted to fit into a catheter hub and through which the needle is received, the nose including an elastomeric gasket integrally associated therewith.

34. The needle guard of claim 19, further comprising a duckbill release mechanism.

35. A needle guard for a catheter insertion device comprising:
an elongated needle guard housing extending between a distal end and a proximal end and defining a space therebetween;
an elongated needle support housing telescopingly received within the space of the needle guard housing, the needle support housing having a distal end and a proximal end defining a fluid path therebetween;
a needle extending distally from the support housing distal end and having a sharp tip extending out beyond the distal end of the guard housing in a first position of the needle with the distal end of the support housing positioned toward the distal end of the guard housing, the needle being enclosed by the guard housing in a second position of the needle with the distal end of the support housing positioned away from the distal end of the guard housing, the needle having a lumen in fluid communication with the fluid path of the support housing; and
the support housing being elongated sufficiently that a proximal portion thereof is spaced closely to the proximal end of the guard housing in at least the first position of the needle so as to provide an elongated flash chamber for improved flashback visual confirmation.
